(19) United States
(12) Patent Application Publication
(10) Pub. No.: US 2018/0369820 A1
(43) Pub. Date: Dec. 27, 2018

(54) CELL SCREENING METHOD

(71) Applicant: FUJIFILM Corporation, Tokyo (JP)

(72) Inventor: Yasuhisa KANEKO, Ashigarakami-gun (JP)

(73) Assignee: FUJIFILM Corporation, Tokyo (JP)

(21) Appl. No.: 16/120,910

(22) Filed: Sep. 4, 2018

Related U.S. Application Data
(63) Continuation of application No. PCT/JP2017/005183, filed on Feb. 13, 2017.

(30) Foreign Application Priority Data
Mar. 28, 2016 (JP) 2016-064092

(51) Int. Cl. B01L 9/00; B01L 3/00; C12Q 1/04; G01N 15/14

(52) U.S. Cl. CPC B01L 9/523; B01L 3/5085; B01L 3/545; C12Q 1/04; G01N 15/14; G01N 33/491; B01L 2300/0851; B01L 2300/168; B01L 2300/0893; G01N 2015/149; B01L 2300/021

(57) ABSTRACT

Provided is a cell screening method, by which only target cells can be treated in a subsequent step. Disclosed is a cell screening method, including a step of sorting out target cells from a plurality of cells into a first tray 19 in which a plurality of containers 17 are arranged into an array shape; a step of imaging the cells that have been sorted out into the containers 17; and a step of separating the containers 17 containing the sorted cells from the first tray 19 and redisposing the containers 17 into a second tray 119 based on an image captured in the step of imaging.

### CROSS REFERENCE TO RELATED APPLICATIONS

This application is a Continuation of PCT International Application No. PCT/JP2017/005183 filed on Feb. 13, 2017, which claims priority under 35 U.S.C § 119(a) to Patent Application No. 2016-064092 filed in Japan on Mar. 28, 2016, all of which are hereby expressly incorporated by reference into the present application.

### BACKGROUND OF THE INVENTION

### 1. Field of the Invention

The present invention relates to a cell screening method, and more particularly, the invention relates to a cell screening method, by which target cells can be efficiently treated and analyzed.

### 2. Description of the Related Art

Regarding a method for obtaining target cells from a plurality of cells, operations of adding dropwise a plurality of cells altogether onto a well slide, dropping the cells into minute wells, imaging the cells, making a judgment, suctioning particular target cells with capillaries, and transferring the target cells into a polymerase chain reaction (PCR) plate or tube, have been carried out. However, this method has problems that the operation of capillaries is difficult, the operations are time-consuming, and the capillaries are expensive.

Furthermore, sorting of target cells by flow cytometry has been carried out. Flow cytometry is carried out by dispersing fine cells in a fluid, causing the fluid to finely flow, optically analyzing individual cells, and performing determination and sorting of cells to be obtained, based on the results of this analysis.

However, in flow cytometry, there are occasions in which cells other than target cells are incorporated into the cells that have been sorted out, and the proportion of the target cells reaches about several ten percents. Therefore, it is inefficient to perform an analysis, or a preliminary treatment for an analysis, on all of the cells that have been sorted out by flow cytometry.

For example, it is described in JP2014-011986A that a cutout line is provided for each row of a multi-well plate, wells of a row of the plate are detached as necessary, and a plurality of kinds of tests are carried out with the wells.

### SUMMARY OF THE INVENTION

The multi-well plate used in JP2014-011986A allows a plurality of kinds of tests to be carried out conveniently by separating the well plate by each row or by each column. Thus, in a case in which a single row or a single column has cells that are not target cells, cells cannot be separated, and it is not possible to perform an analysis efficiently.

The present invention was achieved in view of such circumstances, and it is an object of the invention to provide a cell screening method that enables only target cells to be treated in a subsequent step.

In order to achieve the object described above, the invention provides a cell screening method comprising a step of sorting out target cells from a plurality of cells into a first tray in which a plurality of containers are arranged into an array shape; a step of imaging the cells that have been sorted out into the containers; and a step of separating the containers containing the sorted cells from the first tray and redisposing the containers into a second tray based on an image captured in the step of imaging.

According to the cell screening method of the present invention, the cells that have been sorted out into the first tray are imaged, and based on the image thus captured, the sorted cells are redisposed from the first tray to the second tray, and thereby the second tray can contain only the target cells. Therefore, by performing an analysis or a preliminary treatment for the analysis using the second tray, the time can be shortened compared to the case of performing an analysis or a preliminary treatment for the analysis using the first tray before the step of redisposing, and thus, an analysis of target cells can be carried out efficiently.

According to another aspect of the invention, in the step of sorting, it is preferable that one cell is sorted out into one container.

In a case where a plurality of nucleated cells exist in one container, there may be occasions in which a subsequent genetic analysis may not be carried out. Therefore, in this case, the unavailability of results is checked by imaging, and the container will not be redisposed into the second tray. Therefore, even in a case in which a target cell has been sorted out into the container, the container will not be selected in the step of redisposing the container. By distributing one cell into one container, a container containing a sorted target cell can be reliably selected in the step of redisposing the container.

According to another aspect of the invention, it is preferable that the step of sorting cells is carried out by flow cytometry.

This aspect discloses an example of the step of sorting cells, and an example of the step of sorting cells may be flow cytometry.

According to another aspect of the invention, it is preferable that the first tray includes a plurality of containers and a plate for holding the containers, and the arrangement information of the first tray is described on at least one of the plate or the containers.

According to this aspect, by describing the arrangement information of the first tray on at least one of the plate or the containers, even in a case where a container is redisposed into the second tray, the position of the container on the first tray can be made clear. Therefore, the position of the container and the information of cells can be correlated by managing the information of the cells in the container on the first tray.

According to another aspect of the invention, it is preferable that the arrangement information is described by inscribing characters, or by printing a two-dimensional barcode, on at least one of the plate or the containers.

This aspect represents an example of describing the arrangement information of the first tray, and the aspect can be carried out by inscribing characters or printing a two-dimensional barcode on at least one of the plate or the containers.

According to another aspect of the invention, it is preferable that the plate has a cutting introduction mechanism.

According to this aspect, by providing a cutting introduction mechanism, cutting out of the plate can be made easier in the step of redisposing.

According to another aspect, it is preferable that the system includes a protective sheet for protecting the containers, and the arrangement information of the first tray is described on the protective sheet.

According to this aspect, the information of cells after redisposition can be made clear by describing the arrangement information of the first tray on the protective sheet for protecting the containers.

According to another aspect of the invention, it is preferable that the arrangement information is inscribed with an ink that is transparent and emits fluorescent light under ultraviolet radiation.

According to this aspect, by inscribing the arrangement information of the first tray described on the protective sheet using a transparent ink, the captured image can be prevented from being affected by the ink described on the protective sheet. Also, by adopting an ink that emits fluorescent light under ultraviolet radiation as the transparent ink, the information can be obtained by reading out the fluorescent light.

According to another aspect of the invention, it is preferable that each of the containers has an RFID tag storing the arrangement information.

According to this aspect, by providing the containers with an RFID tag storing the arrangement information, the information of the cells can be made clear even after redisposition. Furthermore, other information can also be stored using an RFID tag.

According to another aspect of the invention, it is preferable that the antenna portion of the RFID tag is disposed in a ring shape around the outer circumference of the container is a container for PCR the container is a container for PCR container.

According to this aspect, by providing the antenna portion for emitting radio waves of the RFID tag disposed in a ring shape around the outer circumference of a container, the antenna portion becoming an impediment in the case of redisposing can be prevented.

According to another aspect of the invention, it is preferable that the container has a flat bottom face and is transparent.

According to this aspect, by producing the shape of the container to have a flat bottom face and producing the container into a transparent container, a satisfactory image can be captured during the step of imaging.

According to another aspect of the invention, it is preferable that the containers are containers for PCR.

According to this aspect, by using containers for PCR as the containers, a PCR treatment can be carried out using the redisposed second tray. Therefore, in the case of performing a PCR treatment, the PCR treatment can be carried out without taking out cells from the containers.

By employing the cell screening method of the invention, identification of target cells can be carried out by sorting out cells having a high potential of being target cells into a first tray and then imaging the cells. Then, after identifying the cells thus sorted out, the containers on the first tray, into which the target cells have been sorted out, are redisposed into a second tray, and thereby the second tray can be arranged as a tray in which only the target cells have been sorted out. Therefore, by performing an analysis or a preliminary treatment for the analysis using the second tray, a subsequent step can be carried out efficiently.

### BRIEF DESCRIPTION OF THE DRAWINGS

FIG. 1 is a schematic configuration diagram illustrating the configuration of an apparatus for imaging cells.

FIG. 2 is a cross-sectional view illustrating the shape of a container.

FIG. 3 is a diagram illustrating an embodiment of a step of redisposing.

FIG. 4 is a diagram explaining another embodiment of the step of redisposing.

FIG. 5 is a cross-sectional view of a tray used in the embodiment illustrated in FIG. 4.

FIG. 6 is a plan view of a plate having the arrangement information described thereon.

FIG. 7 is a plan view illustrating another embodiment of the plate having the arrangement information described thereon.

FIG. 8 is a side view of a container having a protective sheet.

FIG. 9 is a plan view of the container illustrated in FIG. 8.

FIG. 10 is a cross-sectional view of a container having an RFID tag.

FIG. 11 is a plan view of the container illustrated in FIG. 10.

### DESCRIPTION OF THE PREFERRED EMBODIMENTS

Hereinafter, the cell screening method according to the invention will be described using the attached drawings. In the present specification, the symbol “˜” is used to mean that a range includes the numerical values described before and after the symbol as the lower limit and the upper limit.

The cell screening method of the present embodiment includes a step of sorting out target cells from a plurality of cells into a first tray in which a plurality of containers are arranged into an array shape; a step of imaging the cells that have been sorted out into the containers; and a step of separating the containers in which cells have been sorted out from the first tray and redisposing the containers into a second tray based on the image captured in the step of imaging. In the following description, the various steps will be explained.

<<Step of Sorting>>

The step of sorting out target cells from a plurality of cells can be carried out by, for example, flow cytometry. In the flow cytometry, a sample liquid including a material as an object of measurement, such as cells of an object of measurement, is caused to flow toward the center of a laminar flow of a sheath liquid in a flow cell, the material as an object of measurement is irradiated with laser light at an optical detection unit, the scattered light and fluorescent light produced therefrom are measured, and thereby the size, structure, and the like of the material as an object of measurement are measured. The parameters for measurement at the optical detection unit include forward-scattered light, side-scattered light, and fluorescent light; however, the size of the object of measurement can be measured with forward-scattered light, and the structure and the like of the material as an object of measurement can be measured with side-scattered light and fluorescent light.

Then, target cells are sorted out into containers on the first tray by means of a sorting system using the measurement parameters at the optical detection unit. In flow cytometry, the sample/sheath liquid is designed to flow downward from above, and the sample/sheath liquid rushes out, while being in the state of a laminar flow, and drops through the nozzle at the tip of a flow cell. The entire flow cell or the interior of the flow cell is subjected to vertical vibration using a transducer (oscillator) so that sample/sheath liquid discharged out of the flow cell is converted to liquid droplets (drops) from the middle. Based on the sorting conditions using the measurement parameters at the optical detection unit, it is determined whether the cells are cells to be sorted out, and immediately before the sample/sheath liquid becomes liquid droplets, the sample/sheath liquid is charged positively or negatively. Subsequently, the liquid droplets are dropped between two sheets of deflection plates, and positively charged liquid droplets are attracted toward the negative polar plate side, while negatively charged liquid droplets are attracted toward the positive polar plate side. Thus, cells can be sorted out into various containers on the tray (first tray) in the cell capturing unit.

Regarding the method of flow cytometry, the method described above has been described as an example; however, the method of flow cytometry is not limited to the method described above and can be carried out by any method that is generally carried out. Furthermore, the step of sorting is not limited to flow cytometry and can also be carried out by other methods.

However, in regard to the step of sorting, it is difficult to sort target cells with high accuracy, and for example, in regard to flow cytometry, the proportion at which target cells are obtained from among the cells that have been sorted out into the containers is about several ten percents. It is inefficient to subject all the cells that have been sorted out in the step of sorting, to an analysis or a preliminary treatment for analysis, and in the present embodiment, cells other than the target cells are excluded by capturing images after the step of sorting, and redisposing containers from the first tray to a second tray based on the image thus captured.

<<Step of Imaging>>

Next, it is checked whether the cells that have been sorted out are target cells, by imaging the cells sorted out into the containers.

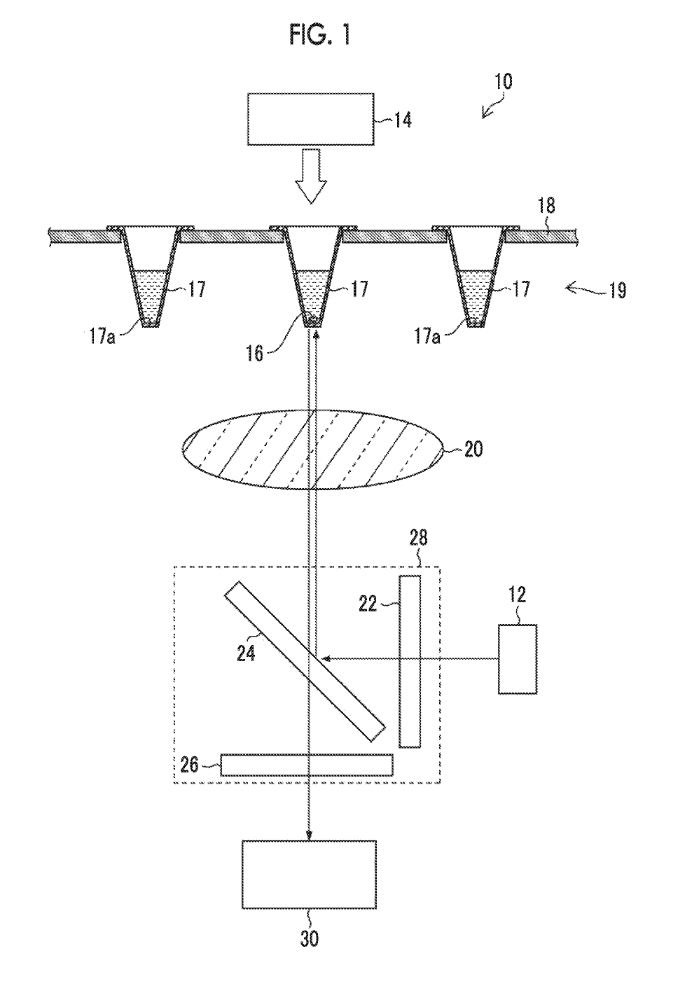

FIG. 1 is a schematic configuration diagram illustrating the configuration of an apparatus for imaging target cells sorted into containers or obtaining optical information from cells. As a preferred embodiment, the apparatus is an analytical apparatus capable of obtaining information of fluorescent light emission from a fluorescent dye used to label sorted cells, by means of an antigen-antibody reaction or the like, or capable of obtaining a light transmission image of cells produced by transmitting visible light.

The analytical apparatus 10 illustrated in FIG. 1 includes an excitation light source apparatus for fluorescence 12, which radiates light for measuring the fluorescent light emitted by object cells; a light source apparatus for bright field 14, which radiates light (visible light) for measuring the light transmitted by cells; a tray (first tray) 19 including containers (wells) 17 accommodating cells 16 that serve as objects of imaging, and a plate 18; a lens 20; a filter group (filter cube) 28 holding an excitation filter 22, a dichroic mirror 24, and a fluorescence filter 26; and an imaging apparatus 30 that captures images of fluorescent light and transmitted light coming from the cells 16.

Regarding the excitation light source apparatus for fluorescence 12, a high pressure mercury lamp, a high pressure xenon lamp, a light emitting diode (LED), a light amplification by stimulated emission of radiation (LASER), or the like can be used. By using these light sources, the wavelength range of the radiated light irradiating the cells 16 is narrowed, and thereby a highly accurate analysis can be carried out more reliably. Regarding the excitation light source apparatus for fluorescence 12, a tungsten lamp, a halogen lamp, a white LED, or the like can be used. Also in the case of using these light sources, the cells 16 can be irradiated with light having a desired wavelength by transmitting only the desired wavelength using the excitation filter 22. Also, regarding the light source apparatus for bright field 14, a light source similar to that for the excitation light source apparatus for fluorescence 12 can be used.

The tray 19 is a sample holder holding the cells 16 that have been sorted out, and includes containers 17 accommodating the cells 16, and a plate 18 holding the containers 17. In the step of sorting, the cells 16 are supplied to the containers 17 together with a culture fluid. In FIG. 1, the containers 17 are described in a simplified manner in order to explain the analytical apparatus 10.

The lens 20 magnifies the fluorescent light emitted by the cells 16 caused by the light output from the excitation light source apparatus for fluorescence 12, and the transmitted light resulting from transmission of the light output from the light source apparatus for bright field 14 by the cells 16. Regarding the lens 20, any lens used for optical measurement can be used.

The filter group 28 includes an excitation filter 22, a dichroic mirror 24, and a fluorescence filter 26. Regarding a specific example of such a filter group 28, it is preferable to use a filter cube, and for example, Zeiss Filter Set 49 (DAPI) can be used. The light radiated from the excitation light source apparatus for fluorescence 12 is such that only the light in a desired wavelength range is transmitted by the excitation filter 22. The light that has permeated the excitation filter 22 is reflected at the dichroic mirror 24 in the direction of the tray 19. The fluorescent light emission from the cells 16 generated by the excited light coming from the excitation light source apparatus for fluorescence 12 passes through the lens 20, the dichroic mirror 24, and the fluorescence filter 26 and is imaged by the imaging apparatus 30. Since the fluorescent light emitted by the excited light has a wavelength bandwidth on the longer wavelength side compared to the excited light, only the fluorescence emission can be transmitted by using the dichroic mirror 24. Furthermore, by using the fluorescence filter 26 that does not transmit excited light but transmits only fluorescent light, imaging by the imaging apparatus 30 based only on the information of the fluorescence emission from the cells 16 is enabled. Therefore, an image can be obtained without having the image to be captured by the imaging apparatus 30 affected by the excited light, and the accuracy of the examination based on the information of fluorescence emission can be improved.

In the fluorescence photography using the light radiated by the excitation light source apparatus for fluorescence 12, since a plurality of information pieces concerning a single cell according to the purpose of examination of cells are acquired, usually, immunostaining is achieved using a plurality of kinds of dyes. In this case, optical information of different wavelengths can be obtained by imaging the fluorescence emitted by each of the dyes of these immunostained cells, using a filter group having transmission characteristics or reflection characteristics that are appropriate for the fluorescence wavelength of each of the dyes. In a case in which the transmitted light of the cells 16 is imaged by the light source apparatus for bright field 14, the image is captured in a state of having the filter group 28 detached. Thereby, the transmitted light can be imaged with the imaging apparatus 30.

The imaging apparatus 30 is not particularly limited as long as the apparatus can image fluorescent light or transmitted light of the cells 16 in the containers 17 on the tray 19, and for example, a charge-coupled device (CCD) camera can be used.

Confirmation of the target cells is carried out using the images obtained by the step of imaging. The confirmation of the target cells based on images can be carried out by screening cells using, for example, the presence or absence of the nucleus, the size of the nucleus, the shape of the nucleus (proportion of the area of the nuclear region with respect to the area of the cytoplasm, and the degree of circularity of the nucleus), the shape of the cell (whether the cell shape is continuous or jagged), the peak value and average value of the luminance of fluorescence, the luminance distribution (whether the cell membrane uniformly emits fluorescence or locally emits intense fluorescence), the degree of absorption of transmitted light at a particular wavelength (distinction between hemoglobin and white blood cell), the spectroscopic characteristics attributed to the difference in the oxygen affinity of hemoglobin (absorption coefficient for the wavelengths of reduced hemoglobin [Hb] and oxidized hemoglobin [HbO₂]), and the like. Regarding specific screening, for example, the standpoints of screening target cells and cells that are different from the target cells, which do not fit the sorting criteria (shape of the cell, absorption of transmitted light, or the like), are determined. Then, the degrees of the standpoints are digitized, and from these measured values, the value range representing the probability of being target cells, and the threshold value representing the value range are determined. This threshold value is determined as the reference value for the screening. As such, a plurality of standpoints for screening are determined in advance, the threshold values for the respective standpoints are determined, and the reference values for the screening are determined. Then, cells that satisfy all of a plurality of the reference values thus determined can be screened as target cells. For example, in a case in which the target cells are nucleated red blood cells or the like, the screening method described in WO2016/021309A or WO2016/021311A can be used.

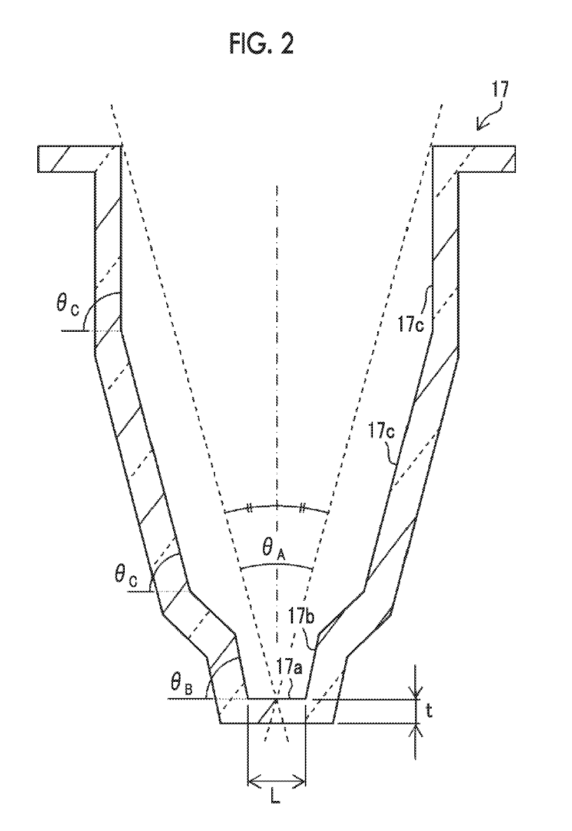

FIG. 2 is a cross-sectional view illustrating a preferred shape of the containers used in the present embodiment. In regard to the analytical apparatus 10 illustrated in FIG. 1, since a container 17 is irradiated with excited light through the back surface side, and the light that has been transmitted through the container 17 and includes information from the cells, such as the fluorescent light from the cells that emit light due to the excited light, is received, conditions such as that the material of the container 17 is transparent, does not emit autofluorescence, and does not scatter light, are required. Furthermore, since the cells 16 are imaged, it is preferable that the bottom face 17a of the container 17 is made flat. By making the bottom face 17a of the container 17 flat, the focus can be placed on the cells 16, and an image analysis of the cells 16 existing on the bottom face 17a can be carried out accurately.

It is also preferable that the shape of the bottom face 17a is a circular shape or a polygonal shape such as a rectangular shape or a higher polygonal shape. Regarding the size of the bottom face 17a, in a case in which a circle circumscribing the bottom face 17a is drawn to approximate the bottom face 17a, it is preferable that the diameter L of the circle is adjusted to be from 0.05 mmϕ to 1 mmϕ, and more preferably from 0.2 mmϕ to 0.5 mmϕ. In FIG. 2, the shape of the bottom face 17a is described as a circular shape. By adjusting the shape and size of the bottom face 17a to the shape and size described above, and by using an objective lens having a magnification ratio of from 5 times to 63 times, the entire bottom face 17a can be imaged to a preferred size of cell image and by imaging one visual field (one-shot imaging). In regard to the step of imaging, for fluorescence imaging, an image of three fluorescent colors needs to be taken, and for bright field imaging, an image of four colors needs to be taken. Furthermore, in a case in which a plurality of images of the bottom face are captured for one color, a multiple of the number of images should be captured, and it takes a long time. By achieving one-shot imaging, capturing and analysis of images can be carried out efficiently.

It is also preferable that lateral faces 17b and 17c of the container 17 are formed in directions that spread away from the bottom face toward the opening of the container. By making the opening of the container 17 wide and making the container narrower toward the bottom face 17a, cells 16 can be made easily introduced into the container 17 and easily guided to the bottom face 17a.

In regard to the lateral faces of the container 17, the lateral face 17b that is in contact with the bottom face 17a is preferably such that at the angle formed by the bottom face 17a and the lateral face 17b, the angle θ_{B} on the lateral face side is from 50° to 80°. By adjusting the angle θ_{B} formed by the bottom face 17a and the lateral face 17b to the range described above, the space formed by the bottom face 17a and the lateral face 17b can be made narrow, and the cells 16 can be submerged into the culture fluid with a small amount of the culture fluid. Furthermore, the depth of the culture fluid inside the container 17 becoming shallow can be prevented, and thus drying of the culture fluid and the cells 16 can be prevented. Also, air bubbles in the culture fluid can be eliminated easily.

As illustrated in FIG. 2, it is preferable that the lateral face is bent in multiple stages. In a case in which the lateral face is bent in multiple stages, the lateral face 17c other than the lateral face 17b that is in contact with the bottom face 17a is preferably such that at the angles formed by the respective lateral faces 17c and the bottom face 17a, the angle θ_{c} on the lateral face side is from 40° to 90°. In a case in which the angle is 40° or higher, the cells can be reliably accommodated to the bottom face without having the cells remaining on the slopes of the lateral faces 17c. In a case in which the angle is 40° or higher, the opening of the container 17 can be made narrow, and the container can be accommodated in a narrow space of the tray 19, which is preferable. Furthermore, in a case in which the lateral face is bent in multiple stages, it is preferable that the angle θ_{c} becomes gradually smaller from the opening toward the bottom face 17a, except for the lateral face 17b that is in contact with the bottom face 17a. By adopting such a configuration, the cells that have been sorted out into the container 17 can be guided easily to the bottom face 17a.

It is also preferable that the angle θ_{A}, which is an angle equivalent to twice the angle formed by a line that connects the center of the bottom face 17a (center of a circle approximating the circumscribing circular shape) and the edge of the opening, and a straight line perpendicular to the bottom face, is less than 45°. By setting the angle θ_{A} to be less than 45°, the opening of the container 17 becoming wide can be prevented, and the space of the tray 19 can be made smaller.

It is preferable that the thickness t of the bottom face 17a of the container 17 is adjusted to be from 0.2 mm to 1 mm. In the step of imaging, imaging is carried out from the side of the bottom face 17a of the container 17; however, in a case in which the thickness of the bottom face 17a is 1 mm or less, the lens 20 can approach the cells 16, and it is preferable. Furthermore, in a case in which the thickness is 0.2 mm or more, the focus of scratches on the outer side of the container 17, or any attached waste, contaminant or the like is shifted from the focal depth and does not affect the image to be captured, and only an image of the cells can be captured, which is preferable. The thickness t of the bottom face 17a is most preferably 0.4 mm.

Regarding the material for the container 17, it is preferable to use a material that can easily transmit light in the step of imaging, and specifically, a material selected from an acrylic resin, polypropylene, and polystyrene can be used. A container produced from such a material preferably has a transmittance at a wavelength of from 350 nm to 800 nm of 60% or higher, more preferably 70% or higher, and even more preferably 80% or higher. According to the invention, the “transmittance” is the value obtained by dividing the transmitted light by the incident light (transmittance=transmitted light/incident light), and for example, in a case in which the luminous flux that has been transmitted when a luminous flux of 100 is incident is 60, the transmittance is calculated to be 60%.

Regarding the external shape of the container 17, a shape enabling mounting of the container on an apparatus with which the treatment of a subsequent step is carried out is preferred. For example, it is preferable that the container is produced as a container for PCR and has a shape that enables mounting on an apparatus performing a PCR treatment and a PCR thermal cycler. By adapting the invention to an apparatus that performs a PCR treatment, a PCR treatment can be carried out using a second tray on which only containers into which target cells have been sorted out are disposed in the step of redisposing as a subsequent step, and therefore, a PCR treatment can be carried out without using glass capillaries for exclusive use. In a case in which the containers can be mounted in a PCR thermal cycler, it is preferable that the gap between the apparatus and the external shape of the container 17 is small. By making the gap between the apparatus and the container 17 small, temperature can be efficiently applied to the container at the time of performing a PCR treatment. Furthermore, containers 17 produced by using the above-described materials also have excellent heat resistance, and even in a case in which the containers are applied to a PCR thermal cycler, a PCR treatment can be carried out without having the containers deteriorated.

<<Step of Redisposing>>

Next, the containers on the first tray 19 are redisposed onto a second tray 119, based on the image thus captured. By redisposing the containers, only those cells for which a genetic analysis is carried out are subjected to a genetic analysis or a preliminary treatment for the analysis is carried out, and thereby the time taken for a genetic analysis can be shortened.

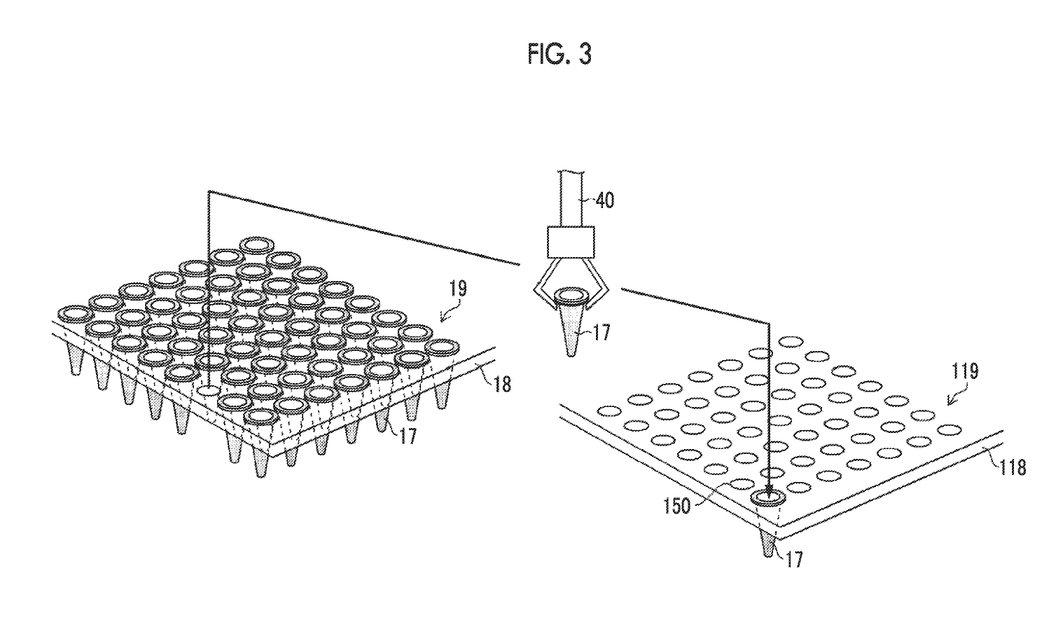

FIG. 3 is a diagram illustrating an embodiment of the step of redisposing. In the containers 17 disposed on the plate 18 of the first tray 19, cells that are considered as target cells are sorted out by the step of sorting. Furthermore, based on the images obtained in the step of imaging, containers 17 containing target cells, which will be redisposed into the second tray 119, are determined. The containers 17 are not adhered to the plate 18, and containers 17 only are conveyed to a well holder 150 formed on a plate 118 by means of, for example, a conveyance mechanism 40. Thereby, only those containers containing target cells can be disposed on the second tray 119.

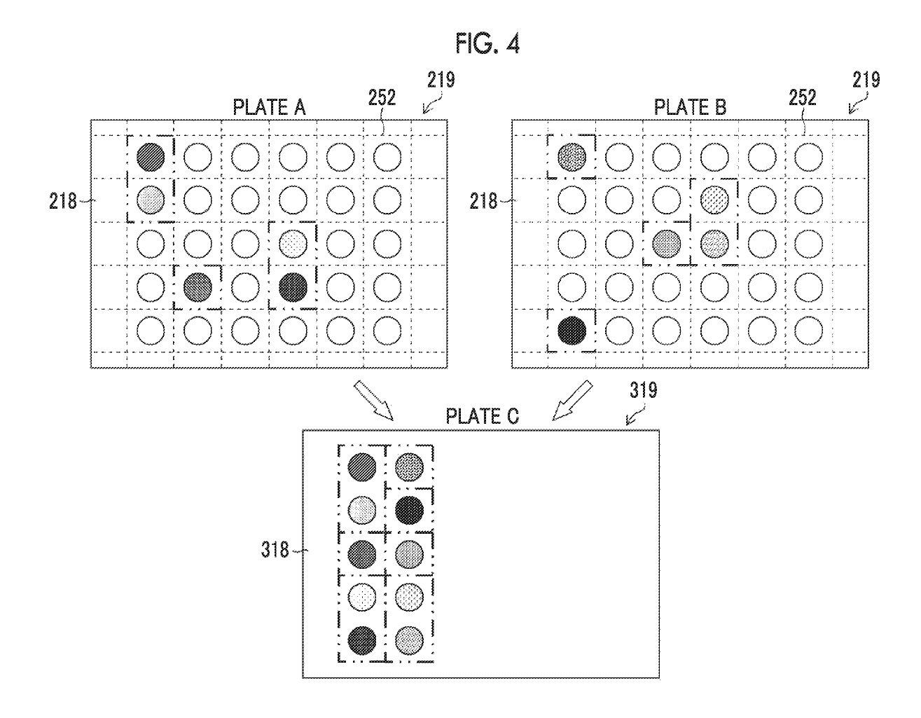

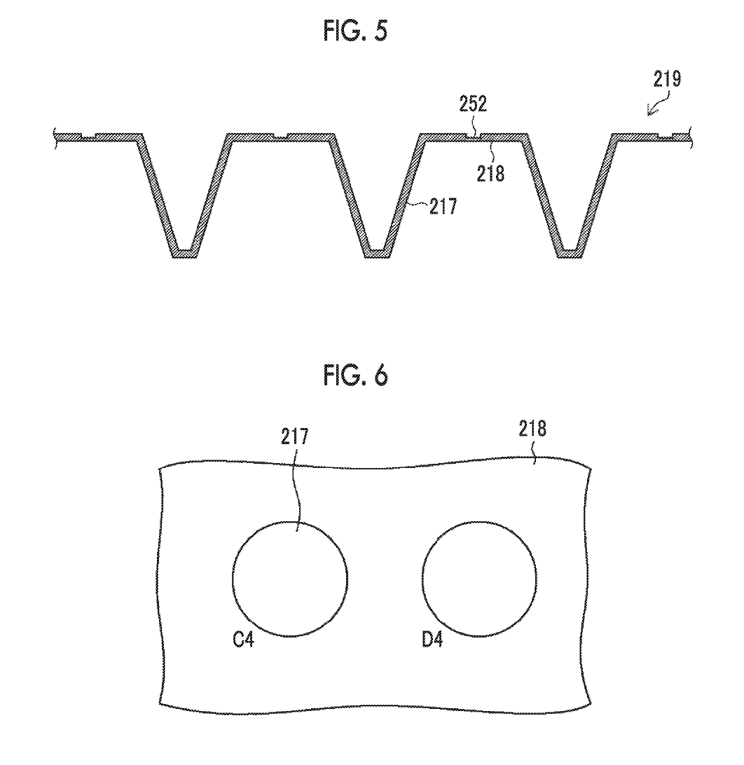

FIG. 4 is a plan view of the plate with which the step of redisposing according to another embodiment is explained, and FIG. 5 is a cross-sectional view of the first tray 219 used in the step of redisposing illustrated in FIG. 4. The step of redisposing as illustrated in FIG. 4 is different from the step of redisposing as illustrated in FIG. 3 from the viewpoint that the first tray 219 is formed such that containers 217 and a plate 218 are integrated.

In a case in which the containers 217 and the plate 218 are formed in an integrated form, it is preferable that the plate 218 is provided with a cutting introduction mechanism 252. Regarding the cutting introduction mechanism 252, a groove, a perforated line, or a printed line provided on the plate 218 can be utilized. After providing the cutting introduction mechanism 252 and determining the containers to be redisposed, the plate 218 is cut out along the cutting introduction mechanism 252 by cutting means, and thereby containers 217 containing target cells can be redisposed into the second tray 319.

In a case in which the plate 218 is cut out and redisposed, for example, the containers can be redisposed as illustrated in FIG. 4. An image analysis was performed for a plate A having 5×6 containers (wells), and there were five target cells. Also, in a plate B, there were five target cells. From the plate A and plate B, the plate 218 is cut out along the cutting introduction mechanism 252 around the containers containing target cells, and the cut-out containers are redisposed on a plate 318 (plate C). Thus, the plate 318 can have the containers containing only target cells.

As such, by redisposing the containers containing target cells into the plate C, only the plate C can be subjected to an analysis or a preliminary treatment for an analysis, and thereby time can be shortened, whereas in conventional methods, the plate A and plate B have to be respectively subjected to an analysis or a preliminary treatment for an analysis. Since containers can be disposed on the plate C, further shortening of the time can be achieved by redisposing the containers containing target cells detached from other first trays (plates) into the plate C.

In the step of redisposing, in a case in which a plurality of cells other than the target cells are included in a container, it is determined that those cells are not the target cells, and these cells are not selected. This is because even in a case in which a PCR treatment of the cells in the container containing a plurality of cells having the nucleus (DNA) is carried out, DNA fragments of a plurality of cells are amplified, and thus, accurate information cannot be obtained by a genetic analysis. On the other hand, in a case in which although other cells are included in a container, it is obvious that these cells do not impede the analysis of the target cells, it is also possible to determine so as to select the container. For instance, it is determined that red blood cells that are not nucleated may be included, while nucleated white blood cells are not selected. Therefore, in the step of sorting, one cell is disposed in one container, and thereby containers containing target cells can be selected reliably in the step of redisposing.

[PCR Treatment]

An example of the preliminary treatment for an analysis may be a PCR treatment. A PCR treatment is a method of amplifying a particular region of a DNA molecule and can be carried out by, for example, the following method. In the present example, the PCR treatment is carried out in order to amplify a particular DNA fragment existing in the target cells. However, the PCR treatment is not limited to the following method. Furthermore, the preliminary treatment for an analysis is also not limited to the PCR treatment, and other treatments may also be carried out.

(Step 1) A reaction liquid (for each plate) obtainable by disrupting or lysing target cells is heated to about 94° C., and the temperature is maintained for 30 seconds to 1 minute. Thus, double-stranded DNA is separated into single strands.

(Step 2) The reaction liquid is rapidly cooled to about 60° C., the single-stranded DNA and primers are heated (annealing) to a predetermined temperature.

(Step 3) The primers are reacted with a DNA polymerase, and the system is heated to a temperature appropriate for the activity of the DNA polymerase, at which separation of the single-stranded DNA and the primers does not occur (about 60° C. to 72° C.). This state is sustained for a time period required for the synthesis of the DNA (may vary depending on the length to be amplified, but usually 1 to 2 minutes).

(Step 4) A particular DNA fragment can be amplified by combining Step 1 to Step 3 into one cycle and repeating the procedure from Step 1 to Step 3. Generally, in a case in which the PCR treatment is carried out for n cycles, a target portion from one double-stranded DNA can be amplified to 2n times. A long DNA chain remains until the end of the process; however, the amount of the remaining DNA can be reduced to an amount that can be neglected, compared to the particular DNA fragment required, usually by performing the treatment for about 20 cycles.

As such, in the PCR treatment, the temperature is increased and decreased within one cycle, and the treatment is usually carried out for about 20 cycles in order to amplify a target portion of the DNA. Therefore, the treatment time takes one hour or longer for each tray. In a case in which only target cells are redisposed into a second tray, and the second tray containing only the target cells is subjected to a preliminary treatment as disclosed by the present embodiment, the time can be shortened to a large extent.

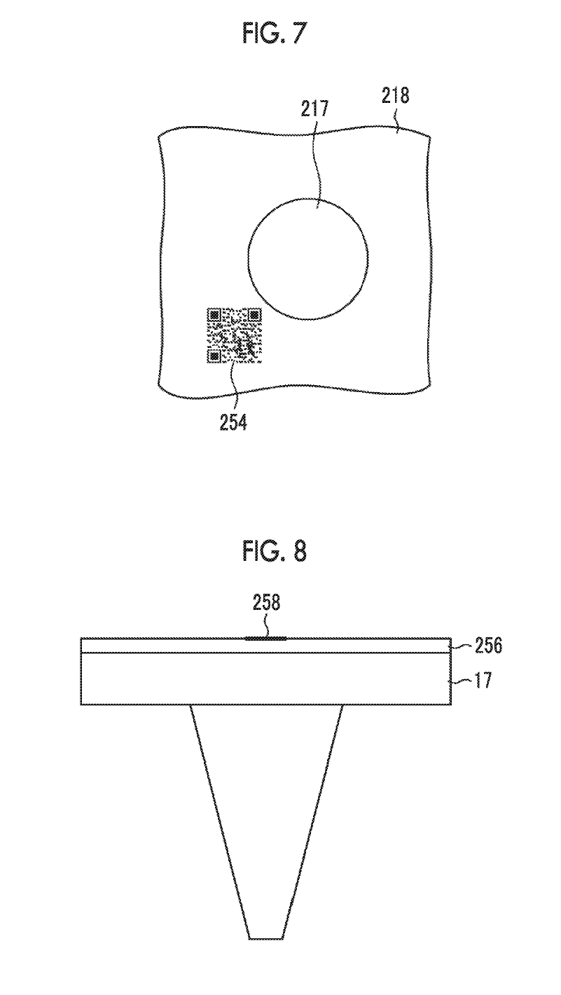

After the step of redisposing, it is preferable that information about the cells in the container is tied up to the container so that the information about the cells can be recognized even after the containers are transferred from the first tray to the second tray. Regarding the method of tying up the information about the cells to the container, for example, as illustrated in FIG. 6, the arrangement information of how the containers 217 are arranged on the plate 218 is inscribed with characters, and the containers are redisposed into the second tray together with the inscribed information. Thus, the information of the cells can be known clearly. Regarding the arrangement information, characters “C4” mean the fourth cell in Row C of the plate 218, and characters “D4” mean the fourth cell in Row D. Furthermore, regarding the method of tying up the information of cells to the container, as illustrated in FIG. 7, the tying up can be carried out by printing QR code (registered trademark) 254 as a two-dimensional barcode on the plate 218. FIG. 6 and FIG. 7 explain the method as embodiments of inscribing the arrangement information on the plate 218 or printing a QR code on the plate 218; however, the place of inscribing or applying the arrangement information is not limited to the plate 218, and the arrangement information may also be inscribed or printed on the containers 217 themselves.

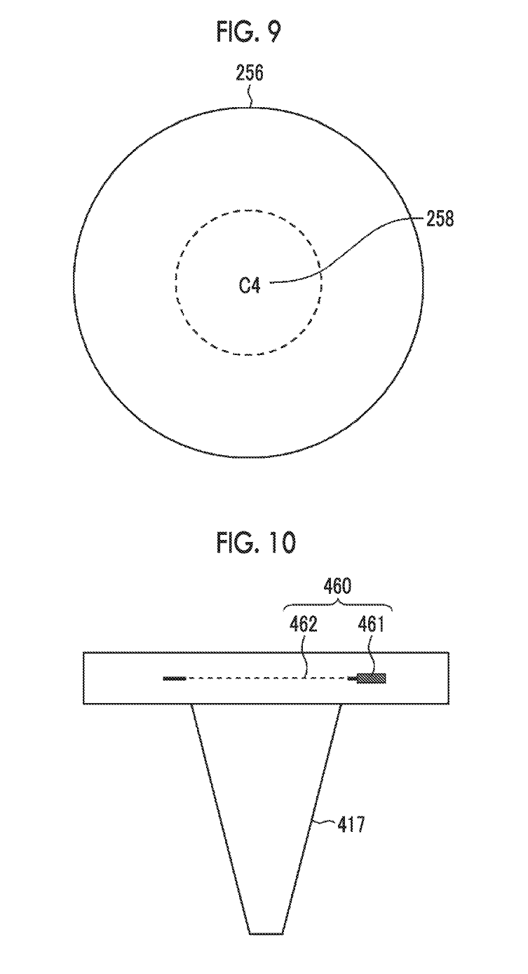

FIG. 8 and FIG. 9 are diagrams explaining other embodiments of tying up the information of cells to the container, and FIG. 8 is a side view of the container 17, while FIG. 9 is a plan view of the container 17. As shown in FIG. 8, the surface of the container 17 containing sorted cells can be usually covered with a protective sheet 256. Therefore, by inscribing the arrangement information of the first tray on the printed surface 258 of this protective sheet 256, the information about cells can be tied up to the container even after the step of redisposing. During the imaging of a bright field for cell observation, as illustrated in FIG. 1, the container 17 is irradiated with light through the opening side. Therefore, in a case in which the information is inscribed on the protective sheet 256, this inscription may cause a reduction in the amount of light or the occurrence of uneven illumination, and there may be a problem in the imaging of the bright field. Therefore, in a case in which inscription is performed on the protective sheet 256, it is preferable that inscription is performed using a fluorescent ink that is transparent under visible light and emits fluorescent light under ultraviolet radiation. In this case, the information can be read out visually by irradiating the information with ultraviolet radiation. Alternatively, in a case in which the inscription is performed in the form of a barcode using a fluorescent ink that fluoresces in red, the fluorescent light can be read out by irradiating the barcode with ultraviolet radiation.

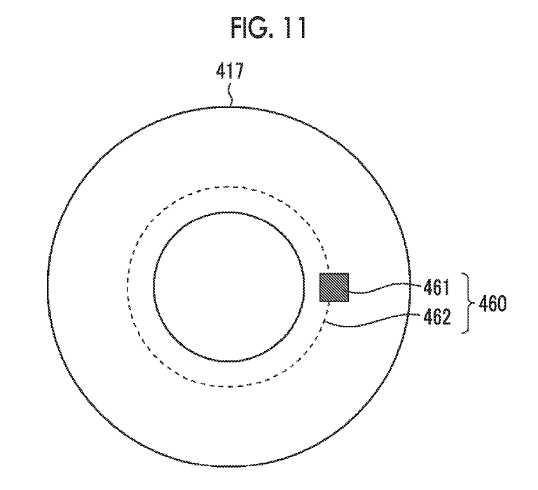

FIG. 10 and FIG. 11 are diagrams explaining still other embodiments of tying up the information of cells to the container, and FIG. 10 is a cross-sectional view of a container 417, while FIG. 11 is a plan view of the container 417. As illustrated in FIG. 10 and FIG. 11, the information about the cells contained in the container 417 can be made clear by providing the container 417 with a radio frequency identifier (RFID) tag 460 storing the arrangement information of the first tray. The RFID tag 460 includes a chip portion 461 retaining the information; and an antenna portion 462 for emitting radio waves. It is preferable that the antenna portion 462 is disposed in a ring shape around the outer circumference of the container 417. By providing the antenna portion 462 disposed in a ring shape, in the case of conveying the container 417 for redisposition, interruption of the conveyance of the container 417 by the antenna portion 462 can be prevented. Furthermore, since the antenna portion 462 is required to have a length corresponding to the frequency of the radio waves to be emitted, the antenna portion 462 is wound around the container 417 in a helical form or is produced into the shape of a sinusoidal wave formed around a circle as a baseline, in order to have a long length. Thus, the antenna portion 462 can be prevented from interrupting the conveyance of the container 417 in the case of redisposing the container.

### EXPLANATION OF REFERENCES

10: analytical apparatus
12: excitation light source apparatus for fluorescence
14: light source apparatus for bright field
16: cell
17, 217, 417: container
17a: bottom face
17b, 17c: lateral face
18, 118, 218, 318: plate
19, 219: first tray
20: lens
22: excitation filter
24: dichroic mirror
26: fluorescence filter
28: filter group (filter cube)
30: imaging apparatus
40: conveyance mechanism
119, 319: second tray
252: cutting introduction mechanism
254: QR code
256: protective sheet
258: printed surface
460: RFID tag
461: chip portion
462: antenna portion

What is claimed is:

1. A cell screening method, comprising:
a step of sorting out target cells from a plurality of cells into a first tray in which a plurality of containers are arranged into an array shape;
a step of imaging the cells that have been sorted out into the containers; and
a step of separating the containers containing the sorted cells from the first tray and redisposing the containers into a second tray based on an image captured in the step of imaging.

2. The cell screening method according to claim 1, wherein the step of sorting is carried out by sorting out one cell into one container.

3. The cell screening method according to claim 1, wherein the step of sorting is carried out by flow cytometry.

4. The cell screening method according to claim 2, wherein the step of sorting is carried out by flow cytometry.

5. The cell screening method according to claim 1, wherein the first tray includes a plurality of containers and a plate for holding the containers, and an arrangement information of the first tray is described on at least one of the plate or the containers.

6. The cell screening method according to claim 2, wherein the first tray includes a plurality of containers and a plate for holding the containers, and an arrangement information of the first tray is described on at least one of the plate or the containers.

7. The cell screening method according to claim 3, wherein the first tray includes a plurality of containers and a plate for holding the containers, and an arrangement information of the first tray is described on at least one of the plate or the containers.

8. The cell screening method according to claim 5, wherein the arrangement information is described by inscribing characters or printing a two-dimensional barcode on at least one of the plate or the containers.

9. The cell screening method according to claim 6, wherein the arrangement information is described by inscribing characters or printing a two-dimensional barcode on at least one of the plate or the containers.

10. The cell screening method according to claim 7, wherein the arrangement information is described by inscribing characters or printing a two-dimensional barcode on at least one of the plate or the containers.

11. The cell screening method according to claim 5, wherein the plate has a cutting introduction mechanism.

12. The cell screening method according to claim 8 wherein the plate has a cutting introduction mechanism.

13. The cell screening method according to claim 1, wherein each of the containers is provided with a protective sheet for protecting the container, and the arrangement information of the first tray is described on the protective sheet.

14. The cell screening method according to claim 2, wherein each of the containers is provided with a protective sheet for protecting the container, and the arrangement information of the first tray is described on the protective sheet.

15. The cell screening method according to claim 3, wherein each of the containers is provided with a protective sheet for protecting the container, and the arrangement information of the first tray is described on the protective sheet.

16. The cell screening method according to claim 13, wherein the arrangement information is inscribed with an ink that is transparent and emits fluorescent light under ultraviolet radiation.

17. The cell screening method according to claim 1, wherein each of the containers has an RFID tag storing the arrangement information.

18. The cell screening method according to claim 17, wherein an antenna portion of the RFID tag is disposed in a ring shape around the outer circumference of the container.

19. The cell screening method according to claim 1, wherein the container has a flat bottom face and is transparent.

20. The cell screening method according to claim 1, wherein the container is a container for PCR.
